(19) United States
(12) Patent Application Publication
(10) Pub. No.: US 2008/0027516 A1
(43) Pub. Date: Jan. 31, 2008

(54) Light modulation system for medical treatment and health care

(75) Inventors: Chien-Jung Wu, Taipei Hsien (TW); Tsung-Ting Sun, Taipei Hsien (TW); Hung-Ta Liao, Taipei Hsien (TW); Tzu-Hsuan Yen, Taipei Hsien (TW); Pao-Shen Chen, Taipei Hsien (TW)

Correspondence Address: BIRCH STEWART KOLASCH & BIRCH, PO BOX 747, FALLS CHURCH, VA, 22040-0747, US

(73) Assignee: EDISON OPTO CORPORATION

(21) Appl. No.: 11/705,131

(22) Filed: Feb. 12, 2007

(30) Foreign Application Priority Data
Jul. 28, 2006 (TW) 95127894

(51) Int. Cl. A61N 5/06

(52) U.S. Cl. 607/88

(57) ABSTRACT

A light modulating system for medical treatment and health care (MTHC) is applied to sense at least one of the physiology conditions of at least one user, and accordingly modulate at least one MTHC light beam. The system comprises a physiology condition parameter sensing unit, a light output parameter calculation unit, and a light modulation unit. The physiology condition parameter sensing unit is applied to sense at least one physiology condition parameter of the user, the light output parameter calculation unit is applied to calculate at least one light output parameter according to the physiology condition parameter, and the light modulation unit is applied to modulate the MTHC light beam according to the light output parameter.

### FIELD OF THE INVENTION

The present invention relates to a light modulation system, and more particularly to a light modulation system being applied to modulate a medical treatment and health care (MTHC) light beam according to at least one physiology condition of an user.

### BACKGROUND OF THE INVENTION

In the prior arts, the light modulation systems are usually applied to illumination. However, it has been discovered that light-waves have effective performance in medical treatment, health care, and beauty treatment after a lot of medical workers have been put numerous efforts in relative academic researches. After that, numerous medical engineers strive to research and develop the optic MTHC system according to the academic research reports.

Thus, following up, we will summarize relative medical research reports, and cite a related prior art to explain the current development of the optic medical treatment and medical treatment (MTHC) system.

Firstly, we summarize the medical research reports relative to the performance of lights with respect to medical treatment, health care, and beauty treatment toward human beings, and list them as follows:
1. Pure white spectrum light:
   Resist the metabolism of melatonin; and
   relax seasonal melancholia.
2. Red light (stimulate sympathetic nerve):
   Resist migraine; and
   enhance strength and arm muscular activation;
   Red light with the wavelength of 633 nm:
   Have well medical performance in smoothing away fine wrinkles, apparent pores and pimples; and
   reduce tumidity after operations to shorten reinstatement periods.
3. Blue light (stimulate parasympathetic nerve):
   Represent relax to reduce anxiousness;
   Blue light with the wavelength of 450 nm:
   Have well medical performance in curing icterus nenatorum; and
   cure arthritis and reduce pain.
4. Combination of red light and blue light:
   Cure intermediate pimples, wherein the blue light is applied for sterilize, and the red light is applied for resisting inflammation.
5. Light approaching to infrared light with the wavelength of 815 nm:
   Promote to heal over wounds.
6. Green light:
   Resist to aging, nourish skin, and lighten scars and fine wrinkles.
7. Yellow light:
   Enhance immunity, and relax and balance sensitivity.
8. Combination of blue light and green light:
   Prevent time differences, and stimulate brain to adjust biological clock.

Through the analyses as summarized above, a prediction can be made, the prescription provided by doctors after finishing medical treatment will include optical prescription to project the MTHC light beam with specified wavelength to a patient. Thus, it is necessary to develop proper light modulation system to catch up with the needs in the future.

Continuously, a further prior art related to the medical treatment light modulating techniques will be discussed further as below. As shown in FIG. 1, a prior medical treatment light filtering system 1 is applied to that of sensing at least one physiology condition of a user 100 to filter a medical treatment light beam and then projecting the medical treatment light beam to the user for some medical operations. The medical treatment light filtering system 1 includes a physiology parameter sensing unit 11, a processing unit 12, a light output unit 13 and a medical treatment light emitter 14, wherein the light output unit 13 includes a light source 131 and a filtering element 132.

When the user 100 operates the medical treatment light filtering system 1, the physiology condition parameter sensing unit 11 senses the physiology condition parameters, such as body temperature, blood pressure and heartbeat of the user 100, and transmits the physiology condition parameters to the processing unit 12 for processing. After the processing unit 12 processes the physiology condition parameters, a waveband parameter is output to the light output unit 13. The filtering element 132 of the light output unit 13 is capable of filtering a medical treatment light beam, corresponding to the waveband parameter, from the light source 131. Finally, the medical treatment light emitter 14 can project the filtered medical treatment light beam to the user 100.

However, people skilled in relative arts can easily realize that, the medical treatment light beam is provided by filtering the light beam projected from the light source 131 via the filtering element 132. Thus, in practice, it is limited by the property of the filtering element that can only modulate light beam in the form of “waveband modulation”. For example, the medical treatment light beam can only be modulated among one of the four wavebands of 600 nm to 610 nm, 610 nm to 620 nm, 620 nm to 630 nm, and 630 nm to 640 nm, but cannot be modulated in a single specified wavelength, such as 633 nm. Meanwhile, for satisfying different requirements of filtering, it is necessary to prepare a plurality of filtering elements 14 as spare parts for substitution.

Moreover, the medical treatment light filtering system 1 will not be capable of projecting a suitable medical treatment light beam when the user 100 suffers from psychological sickness, which can not be presented in the physiology condition parameter(s), such as seasonal melancholia, or has appearance defect(s), such as apparent pores, so that the performance of medical treatment is obviously limited.

### SUMMARY OF THE INVENTION

The problems intended being solved in the present invention and the objects of the present invention are described as follows:

Summarizing above description, as the prior medical treatment light filtering system, it generally exists the problems of the single specified wavelength being not modulated, the plurality of filtering elements being prepared, and medical operations for psychological sickness or appearance defects being not engaged in.

Thus, the primary object of the present invention provides a light modulation system for medical treatment and health care (MTHC) to automatically modulate at least one MTHC light beam with the specified wavelength in accordance with at least one physiology condition parameter, and progressively engage in the actions of medical treatment, health care, and beauty treatment to the user.

Thus, the secondary object of the present invention provides a light modulation system for MTHC, which can automatically modulate the MTHC light beam with the single specified wavelength without preparing any substituted element, such as the filtering elements with different filtering wavebands as mentioned in the background.

Another object of the present invention provides a light modulation system for MTHC further including a MTHC light input unit, which can be input with at least one specified light output parameter, such as wavelength, strength, and MTHC light output time, etc., according to the results of medical treatment, by the user. And the present invention can automatically modulate proper a MTHC light beam corresponding to the specified light output parameters.

Means of the present invention for solving problems:

Means of the present invention for solving the problems as mentioned above provide a light modulation system for MTHC, which can sense at least one physiology condition of at least one user and modulate a MTHC light beam in accordance with the current physiology condition of the user. The light modulation system comprises a physiology condition sensing unit, a light output parameter calculation unit and a light modulation unit. The physiology condition sensing unit is applied to sense at least one physiology condition parameter representing to the physiology condition of the user; the light output parameter calculation unit is applied to calculate at least one light output parameter, such as wavelength, strength, and MTHC light output time, etc., according to the physiology condition; and the light modulation unit is further applied to modulate the MTHC light beam according to the light output parameter.

In a preferred embodiment of the present invention, the light modulation system for MTHC further comprises a MTHC light parameter input unit, so that it is able to be input with the light output parameter by the user according to the results of medical treatment provided by a doctor or the medical common senses of the user. Thus, the present invention can automatically modulate proper MTHC light beam corresponding to the light output parameter.

Effects of the present invention with respect to prior arts:

Making a comparison with the medical treatment light filtering system of the prior arts and the light modulation system for MTHC of the present invention, the filtering concept of the prior arts is dismissed and replaced by a light-mixing concept to modulate the MTHC light beam as disclosed in the present invention. The efficiencies of the present invention not only can precisely and automatically modulate the MTHC light beam, but also can execute the techniques without preparing any substituted elements. Furthermore, the light modulation system for MTHC of the present invention can be operated by the user in accordance with the prescription provided by the doctor or the medical common sense(s) of the user, to modulate proper MTHC light beam to apparently improve the convenience of usage.

The devices, characteristics, and the preferred embodiment of this invention are described with relative figures as follows.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a block diagram illustrating a medical treatment light filtering system provided in accordance with prior arts;

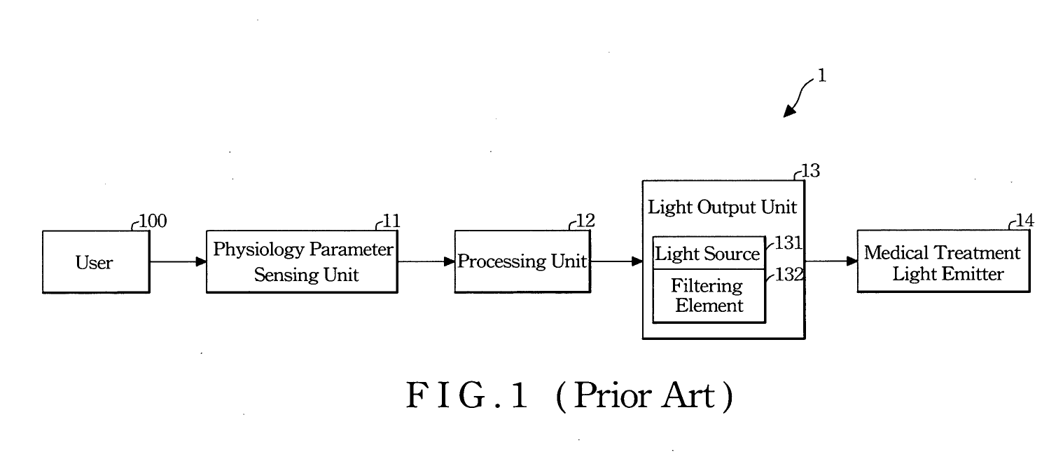

FIG. 2 is a block diagram provided in accordance with a preferred embodiment of the present invention; and

FIG. 3 is a block diagram illustrating a light modulation unit in accordance with the preferred embodiment of the present invention.

### DETAILED DESCRIPTION OF THE PREFERRED EMBODIMENTS

Due to that the light modulation system as provided in accordance with the present invention can be widely applied in the medical treatment and health care (MTHC) fields, the combined applications are too numerous to be enumerated and described so that we only disclose a preferred embodiment for representation.

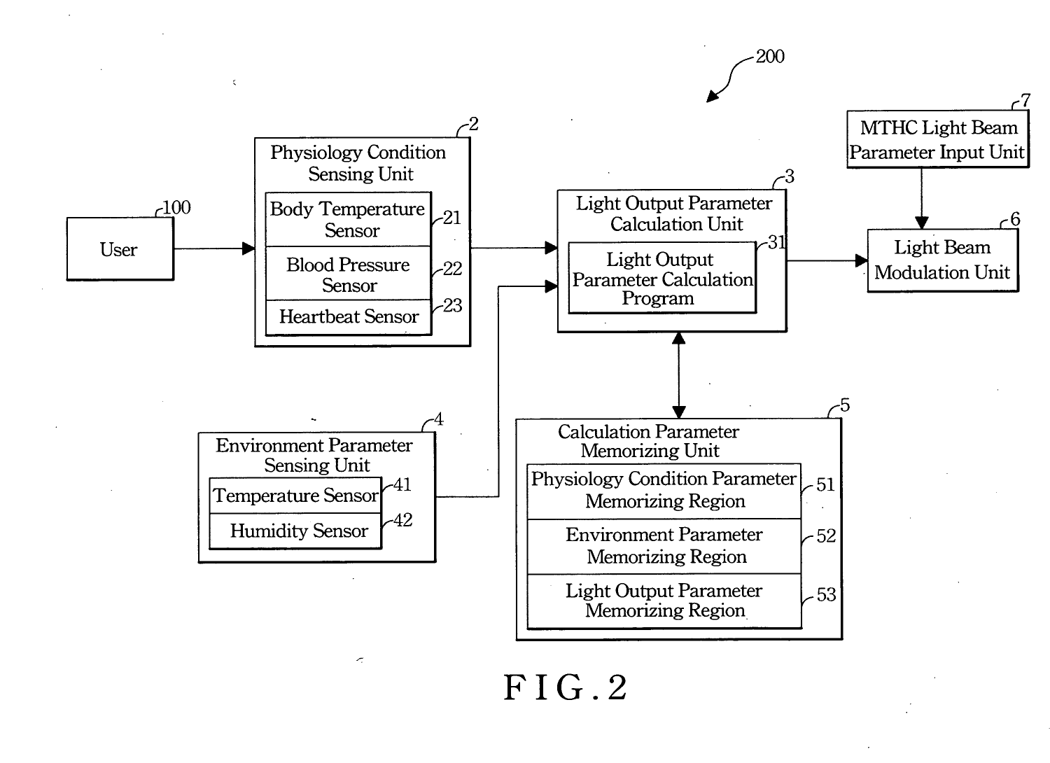

Please refer to FIG. 2, which illustrates a block diagram provided in accordance with a preferred embodiment of the present invention. As shown in FIG. 2, a light modulation system 200 for MTHC is applied for sensing the physiology condition(s) of the user 100 as mentioned above to accordingly modulate at least one proper MTHC light beam. The light modulation system 200 includes a physiology condition sensing unit 2, a light output parameter calculation unit 3, an environment parameter sensing unit 4, a calculation parameter memorizing unit 5, a light beam modulation unit 6, and an MTHC light beam parameter input unit 7.

The physiology condition sensing unit 2 includes a body temperature sensor 21, a blood pressure sensor 22, and a heartbeat sensor 23 for respectively sensing the physiology condition parameters, representing the current physiology conditions of the user, such as the body temperature, the blood pressure, and the heartbeat (or the pulse). The light output parameter calculation unit 3 includes a light output parameter calculation program 31. The environment parameter sensing unit 4 includes a temperature sensor 41 and a humidity sensor 42 to respectively sense at least one environment parameter, such as temperature and humidity represented by the relative humidity in percentage, around the user 100. The calculation parameter memorizing unit 5 includes a physiology condition parameter memorizing region 51, an environment parameter memorizing region 52, and a light output parameter memorizing region 53 for respectively memorizing the physiology condition parameter, the environment parameter, and the light output parameter.

When the user 100 or a doctor uses the light modulation system 200 to engage in a medical treatment, health care or beauty treatment action, several physiology condition parameters of the user or a patient can be sensed through the physiology condition parameter sensing unit 2, and the environment parameters around the user 100 can be sensed by the environment parameter sensing unit 4 as well. Then, the sensed physiology condition parameters and environment parameters are transmitted to the light output calculation unit 3, and also respectively transmitted to and saved in the physiology condition parameter memorizing region 51 and the environment parameter memorizing region 52.

Next, the light output parameter calculation program 31 of the light output parameter calculation unit 3 can automatically calculate at least one light output parameter, including at least one of the wavelength, the strength, and the output time of the MTHC light beam, and transmit the light output parameter to the light modulation unit 6. Finally, the light modulation unit 6 can automatically modulate the proper MTHC light beam in accordance with the light output parameter to engage in the action(s) of medical treatment, health care, and/or beauty treatment to the user 100.

In practice, although some psychology sicknesses can be diagnosed from the variations of physiology conditions to gain the elementary diagnosis results, there are still many sicknesses cannot be diagnosed from the variations of physiology conditions yet. To solve the problem, the light modulation system 200, in accordance with the preferred embodiment of the present invention, further provides the MTHC light parameter input unit 7, so that the user 100 still can directly input the light output parameter, according to the prescription provided by the doctor or the medical common senses of the user, via the MTHC light parameter input unit 7 to modulate the proper MTHC light beam for engaging in the action(s) of medical treatment, health care, and/or beauty treatment.

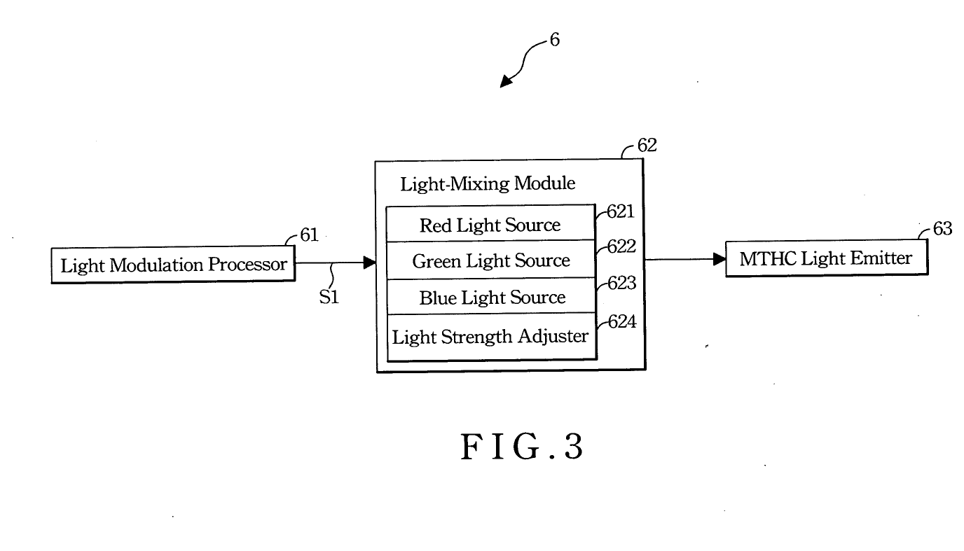

About the light modulation unit 6, there are some more applications and will be disclosed further in the details of the preferred embodiment of the present invention. Referring to FIG. 3, which is a block diagram illustrating the light modulation unit in accordance with the preferred embodiment of the present invention. As shown in FIG. 3, the light modulation unit 6 includes a light modulation processor 61, a light-mixing module 62, and a MTHC light emitter 63, wherein the light-mixing module 62 includes a plurality of light sources for respectively projecting the plurality of essential light beams. In the preferred embodiment of the present invention, the light sources are visible-light sources for projecting visible light beams served as the essential light beams. In another words, the visible light sources of the present invention include a red light source 621, a green light source 622, and a blue light source 623 for respectively projecting a red light beam, a green light beam and a blue light beam. By the way, the light-mixing module includes a light strength adjuster 624 as well.

Following the description related to FIG. 2, when the light output parameter(s) is/are input to the light modulation unit 6, the light output parameter(s) can be transmitted to the light modulation processor 61 to engage in processing and accordingly transmitting a light modulation signal S1 to the light-mixing module 62. According to the light modulation signal S1, the light strength adjuster 624 can adjust the strength and the output time of the red light beam, the green light beam, and the blue light beam respectively projected from the red light source 621, the green light source 622, and the blue light source 623 to modulate the proper MTHC light beam. Finally, the MTHC light emitter 63 can project the MTHC light beam to at least one selected position, where is usually a specified portion of the user 100, to engage in the action(s) of medical treatment, health care, and/or beauty treatment.

In the preferred embodiment, the red light source 621, the green light source 622, and the blue light source 623 are submitted to be a red light emitting diode (LED), a green LED, and a blue LED, so that the MTHC light beam can be modulated in an economic way. Additionally, the light-mixing module 62 can further include a plurality of invisible-light sources, such as an infrared light source and an ultraviolet light source, for respectively the plurality of invisible light beams, such as an infrared light beam and an ultraviolet light beam, served as the essential light beams to progress the modulation of MTHC light beam, so that the MTHC light beam itself also can be an invisible light, which is not limited in visible light as mentioned in the preferred embodiment of the present invention.

After reading above description, people skilled in the fields of optic medical treatment and light modulation technique can easily realize that the light modulation system for MTHC of the present invention has dismissed the concept of light filtering in prior arts and replaced by a concept of light-mixing to modulate the MTHC light beam. The efficiencies of the present invention not only can precisely and automatically modulate the MTHC light beam, but also can execute the techniques without preparing any substituted elements. Furthermore, the light modulation system for the MTHC of the present invention can be operated, in accordance with the prescription provided by the doctor or the user, to modulate proper MTHC light beam.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A light modulation system for medical treatment and health care (MTHC) being applied to sense at least one of a plurality of physiology conditions of at least one user to accordingly modulate at least one MTHC light beam, and comprising:
a physiology condition parameter sensing unit for sensing at least one physiology condition parameter;
a light output parameter calculation unit for calculating at least one light output parameter according to the physiology condition parameter; and
a light modulation unit for modulating the MTHC light beam according to the light output parameter.

2. The light modulation system for MTHC as claimed in claim 1, further comprising a calculation parameter memorizing unit, which comprises a physiology condition memorizing region for memorizing the physiology condition parameter sensed by the physiology condition parameter sensing unit.

3. The light modulation system for MTHC as claimed in claim 1, wherein the physiology condition parameter sensing unit comprises a body temperature sensor for sensing a body temperature of the user as the physiology condition parameter.

4. The light modulation system for MTHC as claimed in claim 1, wherein the physiology condition parameter sensing unit comprises a blood pressure sensor for sensing a blood pressure of the user as the physiology condition parameter.

5. The light modulation system for MTHC as claimed in claim 1, wherein the physiology condition parameter sensing unit comprises a heartbeat sensor for sensing a heartbeat of the user as the physiology condition parameter.

6. The light modulation system for MTHC as claimed in claim 1, further comprising an environment parameter sensing unit for sensing at least one environment parameter around the user.

7. The light modulation system for MTHC as claimed in claim 6, wherein the light output calculation unit further comprises a light output parameter calculation program for calculating the light output parameter according to the physiology condition parameter and the environment parameter.

8. The light modulation system for MTHC as claimed in claim 6, further comprising a calculation parameter memorizing unit, which comprises an environment parameter memorizing region for memorizing the environment parameter sensed by the environment parameter sensing unit.

9. The light modulation system for MTHC as claimed in claim 6, wherein the environment parameter sensing unit comprises a temperature sensor for sensing a temperature around the user as the environment parameter.

10. The light modulation system for MTHC as claimed in claim 6, wherein the environment parameter sensing unit comprises a humidity sensor for sensing a humidity around the user as the environment parameter.

11. The light modulation system for MTHC as claimed in claim 1, wherein the light modulation unit comprises an MTHC light emitter for projecting the MTHC light beam to at least one selected position.

12. The light modulation system for MTHC as claimed in claim 1, wherein the light modulation unit comprises a light modulation processor for processing the light output parameter to accordingly generate a light modulation signal.

13. The light modulation system for MTHC as claimed in claim 12, wherein the light modulation unit comprises a light-mixing module comprising:
a plurality of light sources for respectively projecting a plurality of essential light beams;
at least one strength modulator for respectively modulating a plurality of strengths of the plurality of essential light beams in accordance with the light modulation signal to accordingly mix the essential lights to modulate the MTHC light beam.

14. The light modulation system for MTHC as claimed in claim 13, wherein the light sources comprises at least one invisible-light source for projecting a invisible light beam as one of the essential light beams.

15. The light modulation system for MTHC as claimed in claim 14, wherein the invisible-light source is an infrared light source for projecting an infrared light beam as the invisible light beam.

16. The light modulation system for MTHC as claimed in claim 14, wherein the invisible-light source is an ultraviolet light source for projecting an ultraviolet light beam as the invisible light beam.

17. The light modulation system for MTHC as claimed in claim 13, wherein the light sources comprises at least one visible-light source for projecting a visible light beam as one of the essential light beams.

18. The light modulation system for MTHC as claimed in claim 17, wherein the visible-light source is a red light source for projecting a red light beam as the visible light beam.

19. The light modulation system for MTHC as claimed in claim 18, wherein the red light source is a red light emitting diode (LED).

20. The light modulation system for MTHC as claimed in claim 17, wherein the visible-light source is a green light source for projecting a green light beam as the visible light beam.

21. The light modulation system for MTHC as claimed in claim 20, wherein the green light source is a green LED.

22. The light modulation system for MTHC as claimed in claim 17, wherein the visible-light source is a blue light source for projecting a blue light beam as the visible light beam.

23. The light modulation system for MTHC as claimed in claim 22, wherein the blue light source is a blue LED.

24. The light modulation system for MTHC as claimed in claim 1, further comprising an MTHC light parameter input unit for the user directly input the light output parameter to the light modulating unit to modulate the MTHC light beam in accordance with the light output parameter.

25. The light modulation system for MTHC as claimed in claim 1, wherein the light output parameter is a wavelength of the MTHC light beam.

26. The light modulation system for MTHC as claimed in claim 1, wherein the light output parameter is a strength of the MTHC light beam.

27. The light modulation system for MTHC as claimed in claim 1, wherein the light output parameter is an output time of the MTHC light beam.
